(19) United States
(12) Patent Application Publication
(10) Pub. No.: US 2023/0210482 A1
(43) Pub. Date: Jul. 6, 2023

(54) MEDICAL DEVICES AND METHODS THEREOF

(71) Applicant: SHANGHAI UNITED IMAGING RESEARCH INSTITUTE OF INTELLIGENT IMAGING, Shanghai (CN)

(72) Inventor: Yanqi HUANG, Shanghai (CN)

(73) Assignee: SHANGHAI UNITED IMAGING RESEARCH INSTITUTE OF INTELLIGENT IMAGING, Shanghai (CN)

(21) Appl. No.: 18/146,368

(22) Filed: Dec. 24, 2022

(30) Foreign Application Priority Data
Dec. 30, 2021 (CN) 202111682404.X

(51) Int. Cl. A61B 6/00; A61B 6/03

(52) U.S. Cl. CPC A61B 6/4417; A61B 6/037; A61B 6/5217; A61B 6/5247

(57) ABSTRACT

The present disclosure provides medical devices and methods thereof. The medical device may include a housing, a positron emission tomography (PET) detector module, and a radio frequency (RF) coil. The housing may form a scanning tunnel for accommodating a subject. The PET detector module may be arranged along a circumference of the scanning tunnel. The RF coil may be arranged along the circumference of the scanning tunnel. The RF coil may include a first RF coil and a second RF coil. The first RF coil and the second RF coil may be disposed coaxially around an axial direction of the scanning tunnel. A projection of the second RF coil along a radial direction of the scanning tunnel may cover at least a portion of a gap of the first RF coil.

### CROSS-REFERENCE TO RELATED APPLICATIONS

This application claims priority to Chinese Patent Application No. 202111682404.X, filed on Dec. 30, 2021, the contents of which are incorporated herein by reference.

### TECHNICAL FIELD

The present disclosure generally relates to imaging technology, and more particularly, relates to medical devices and methods thereof.

### BACKGROUND

A positron emission tomography-magnetic resonance (PET-MR) device can perform PET imaging and MR imaging on a subject simultaneously. Therefore, functional imaging and anatomical imaging are combined. Accordingly, the PET-MR device can provide more information than a single modality device (e.g., a PET device or an MR device), thereby can be used to accurately determine a position of a lesion in the subject. However, the PET imaging and the MR imaging may interact with each other, which reduces the accuracy of the PET imaging and the MR imaging and further reduce the imaging quality. Therefore, it is desirable to provide improved PET-MR devices and methods thereof, which can efficiently increase the accuracy of PET imaging and MR imaging.

### SUMMARY

In one aspect of the present disclosure, a medical device is provided. The medical device may a housing, a positron emission tomography (PET) detector module, and a radio frequency (RF) coil. The housing may form a scanning tunnel for accommodating a subject. The PET detector module may be arranged along a circumference of the scanning tunnel. The PET detector module may be configured to detect radiation photons emitted from the subject during a scan. The RF coil may be arranged along the circumference of the scanning tunnel. The RF coil may be configured to receive magnetic resonance (MR) signals related to the subject during the scan. The RF coil may include a first RF coil and a second RF coil. The first RF coil and the second RF coil may be disposed coaxially around an axial direction of the scanning tunnel. A projection of the second RF coil along a radial direction of the scanning tunnel may cover at least a portion of a gap of the first RF coil.

In some embodiments, the second RF coil may be detachably mounted in the medical device.

In some embodiments, the first RF coil may be disposed inside the housing, and the second RF coil may be disposed on an inner surface of the housing.

In some embodiments, the medical device may further include one or more positioning components configured to fix the second RF coil on the inner surface of the housing.

In some embodiments, at least one of the one or more positioning components may include an interface used for current conduction and signal transmission of the second RF coil.

In some embodiments, the housing may include a first housing and a second housing disposed coaxially around the axial direction of the scanning tunnel, the second housing may form the scanning tunnel, the first RF coil may be disposed inside the first housing, and the second RF coil may be disposed inside the second housing.

In some embodiments, a coil parameter of the second RF coil may be determined based on a scanning condition of the subject, the coil parameter of the second RF coil including at least one of a location, a size, a thickness, or a type of the second RF coil.

In some embodiments, the first RF coil and the second RF coil may be integrated in the medical device.

In some embodiments, the second RF coil may include a preamplifier disposed outside a field of view (FOV) of the PET detector module.

In another aspect of the present disclosure, a method is provided. The method may be implemented on a computing device having at least one processor and at least one storage device. The method may include determining, based on a subject to be scanned, one or more scanning parameters of a medical device. The medical device may include a housing, a positron emission tomography (PET) detector module, and a radio frequency (RF) coil. The housing may form a scanning tunnel for accommodating a subject. The PET detector module may be arranged along a circumference of the scanning tunnel. The RF coil may be arranged along the circumference of the scanning tunnel. The RF coil may include a first RF coil and a second RF coil. The first RF coil and the second RF coil may be disposed coaxially around an axial direction of the scanning tunnel. A projection of the second RF coil along a radial direction of the scanning tunnel may cover at least a portion of a gap of the first RF coil. The method may include obtaining PET data and magnetic resonance (MR) data collected by the medical device in a scan of the subject under the one or more scanning parameters. The PET data may be collected by the PET detector module, and the MR data may be collected by the RF coil. Further, the method may include generating, based on the PET data and the MR data, a fused image of the subject.

In some embodiments, the determining, based on a subject to be scanned, one or more scanning parameters of a medical device may include determining a scanning posture of the subject on the medical device.

In some embodiments, the second RF coil may be fixed to the medical device before the subject is injected a PET imaging agent.

In some embodiments, the method may further include determining, based on the fused image, a position of a lesion of the subject.

In still another aspect of the present disclosure, a system is provided. The system may include at least one storage device including a set of instructions; and at least one processor configured to communicate with the at least one storage device. When executing the set of instructions, the at least one processor may be configured to direct the system to perform operations. The operations may include determining, based on a subject to be scanned, one or more scanning parameters of a medical device. The medical device may include a housing, a positron emission tomography (PET) detector module, and a radio frequency (RF) coil. The housing may form a scanning tunnel for accommodating a subject. The PET detector module may be arranged along a circumference of the scanning tunnel. The RF coil may be arranged along the circumference of the scanning tunnel. The RF coil may include a first RF coil and a second RF coil. The first RF coil and the second RF coil may be disposed coaxially around an axial direction of the scanning tunnel. A projection of the second RF coil along a radial direction of the scanning tunnel may cover at least a portion of a gap of the first RF coil. The operations may include obtaining PET data and magnetic resonance (MR) data collected by the medical device in a scan of the subject under the one or more scanning parameters. The PET data may be collected by the PET detector module, and the MR data may be collected by the RF coil. Further, the operations may include generating, based on the PET data and the MR data, a fused image of the subject.

In some embodiments, the operations may further include determining, based on the fused image, a position of a lesion of the subject.

In some embodiments, the second RF coil may be detachably mounted in the medical device.

In some embodiments, the first RF coil may be disposed inside the housing, and the second RF coil may be disposed on an inner surface of the housing.

In some embodiments, the medical device may further include one or more positioning components configured to fix the second RF coil on the inner surface of the housing.

In some embodiments, a coil parameter of the second RF coil may be determined based on a scanning condition of the subject, the coil parameter of the second RF coil including at least one of a location, a size, a thickness, or a type of the second RF coil.

In some embodiments, the first RF coil and the second RF coil may be integrated in the medical device.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2A is a schematic diagram illustrating an exemplary medical device according to some embodiments of the present disclosure;

FIG. 2B is a schematic diagram illustrating another exemplary medical device according to some embodiments of the present disclosure;

FIG. 3A is a schematic diagram illustrating a plan view of an exemplary MRI component according to some embodiments of the present disclosure;

FIG. 3B is a schematic diagram illustrating a plan view of an exemplary medical device according to some embodiments of the present disclosure;

FIG. 4 is a schematic diagram illustrating an exemplary RF coil according to some embodiments of the present disclosure;

FIG. 5 is a schematic diagram illustrating an exemplary RF coil according to some embodiments of the present disclosure;

FIG. 6A is a schematic diagram illustrating an exemplary RF coil according to some embodiments of the present disclosure;

FIG. 6B is a schematic diagram illustrating an exemplary RF coil according to some embodiments of the present disclosure;

FIG. 7 is a schematic diagram illustrating an exemplary RF coil according to some embodiments of the present disclosure;

FIG. 8 is a schematic diagram illustrating an exemplary medical device according to some embodiments of the present disclosure;

FIG. 9 is a block diagram illustrating an exemplary processing device according to some embodiments of the present disclosure; and

FIG. 10 is a flowchart illustrating an exemplary process for generating a fused image of a subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections, or assembly of different levels in ascending order. However, the terms may be displaced by another expression if they achieve the same purpose.

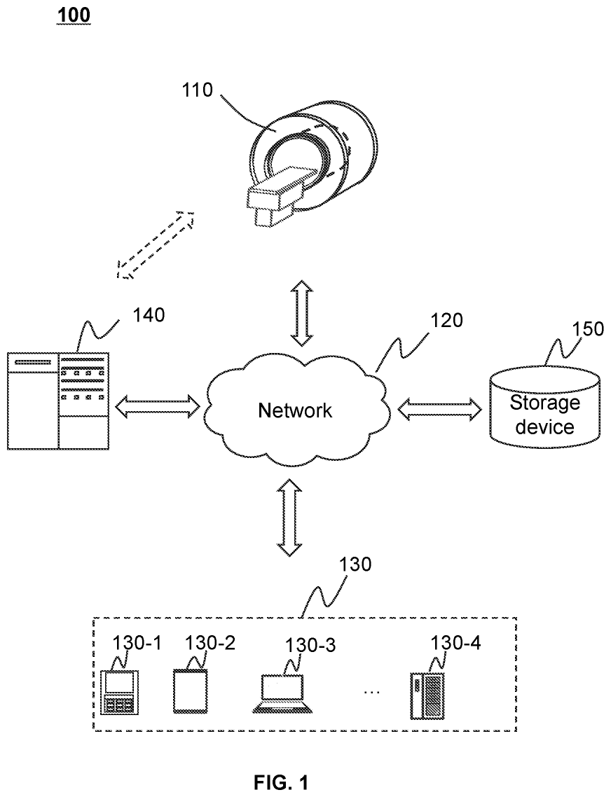

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or other storage devices. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., a processing device 140 as illustrated in FIG. 1) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included in programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. For example, the expression “A and/or B” includes only A, only B, or both A and B. The character “/” includes one of the associated listed terms. The term “multiple” or “a/the plurality of” in the present disclosure refers to two or more. The terms “first,” “second,” and “third,” etc., are used to distinguish similar objects and do not represent a specific order of the objects.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

A PET-MR device can be used to scan a subject to acquire PET data and MR data of the subject. A fused image is generated based on the acquired PET data and MR data for diagnosis and/or radiotherapy (e.g., a stereotactic radiosurgery operation, a precise radiotherapy, etc.). For example, anatomical information (e.g., a position, an area, a range, a relationship with surrounding tissues and vital organs, etc.) of a lesion (e.g., a tumor) may be determined based on the fused image, and such information can be used in treatment planning (e.g., for determining an outline of a target, a radiation dose, a radiotherapy plan, etc.). Therefore, an accuracy of data acquisition of the PET-MR device needs to be ensured and/or improved.

The present disclosure relates to a medical device. The medical device may include a housing, a PET detector module, and a radio frequency (RF) coil. The housing may form a scanning tunnel for accommodating a subject. The PET detector module may be arranged along a circumference of the scanning tunnel. The PET detector module may be configured to detect radiation photons emitted from the subject during a scan. The RF coil may be arranged along the circumference of the scanning tunnel. The RF coil may be configured to receive magnetic resonance (MR) signals related to the subject during the scan. The RF coil may include a first RF coil and a second RF coil, wherein the first RF coil and the second RF coil are disposed coaxially around an axial direction of the scanning tunnel, and a projection of the second RF coil along a radial direction of the scanning tunnel covers at least a portion of a gap of the first RF coil.

By introducing the second RF coil, the medical device may acquire more MR signals related to the subject than a medical device that only includes the first RF coil, which can improve a signal-to-noise ratio and the imaging quality of MR imaging. Further, since the signal-to-noise ratio of the MR imaging is improved, a thickness of the first RF coil and/or the second RF coil can be reduced, which can reduce attenuations of the radiation photons emitted from the subject, thereby improving the imaging quality of PET imaging. Therefore, the imaging quality of the medical device may be improved, and a fused image with an improved imaging quality may be generated based on the radiation photons and the MR signals.

FIG. 1 is a schematic diagram illustrating an exemplary imaging system 100 according to some embodiments of the present disclosure. As shown in FIG. 1, the imaging system 100 may include a medical device 110, a network 120, one or more terminals 130, a processing device 140, and a storage device 150. In some embodiments, the medical device 110, the processing device 140, the storage device 150, and/or the terminal(s) 130 may be connected to and/or communicate with each other via a wireless connection, a wired connection, or a combination thereof. The connection between the components in the imaging system 100 may be variable. Merely by way of example, the medical device 110 may be connected to the processing device 140 through the network 120, as illustrated in FIG. 1. As another example, the medical device 110 may be connected to the processing device 140 directly. As a further example, the storage device 150 may be connected to the processing device 140 through the network 120, as illustrated in FIG. 1, or connected to the processing device 140 directly.

The medical device 110 may be configured to generate or provide image data by scanning a subject or at least a part of the subject. In some embodiments, the medical device 110 may include a multi-modality imaging device. Exemplary multi-modality imaging devices may include a positron emission tomography-magnetic resonance imaging (PET-MRI) device, a single-photon emission computed tomography-magnetic resonance imaging (SPECT-MRI) device, etc. The multi-modality imaging device may perform multi-modality imaging simultaneously or in sequence. For example, the PET-MRI device may generate MRI data and PET data simultaneously or in sequence.

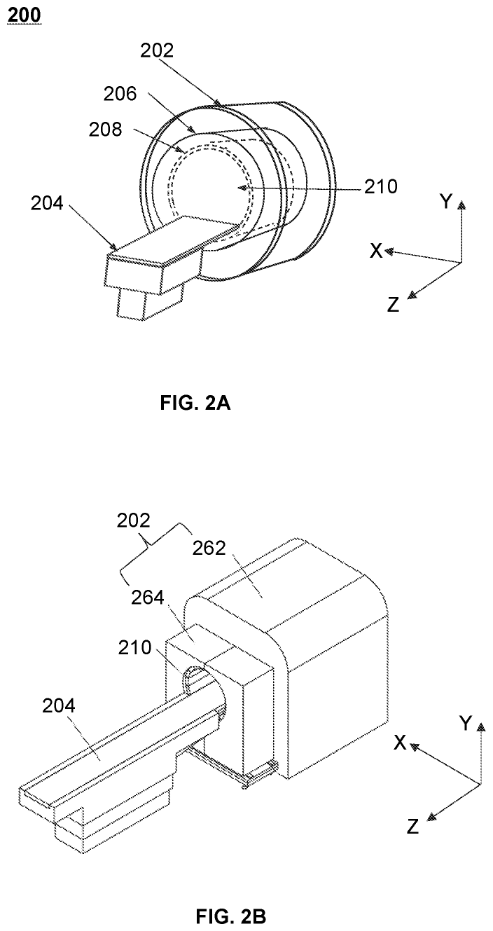

Merely by way of example, the medical device 110 may be a PET-MRI device. The PET-MRI device may refer to a medical device that can perform PET imaging and MR imaging on the subject. For example, the medical device 110 may include a PET detector module and a radio frequency (RF) coil. The PET detector module may be configured to detect radiation photons emitted from the subject during a scan, and the RF coil may be configured to receive MR signals related to the subject during the scan. More descriptions regarding the PET-MRI device may be found elsewhere in the present disclosure. See, e.g., FIGS. 2A-8, and relevant descriptions thereof.

The subject may include patients or other experimental subjects (e.g., experimental mice or other animals). In some embodiments, the subject may be a patient or a specific portion, organ, and/or tissue of the patient. For example, the subject may be a patient to be scanned by the medical device 110. As another example, the subject may include the head, the neck, the thorax, the heart, the stomach, a blood vessel, soft tissue, a tumor, nodules, or the like, or any combination thereof. In some embodiments, the subject may be non-biological. For example, the subject may include a phantom, a man-made object, etc. The terms “object” and “subject” are used interchangeably in the present disclosure.

The network 120 may include any suitable network that can facilitate the exchange of information and/or data for the imaging system 100. In some embodiments, one or more components (e.g., the medical device 110, the terminal(s) 130, the processing device 140, the storage device 150, etc.) of the imaging system 100 may communicate information and/or data with one or more other components of the imaging system 100 via the network 120. For example, the processing device 140 may obtain image data from the medical device 110 via the network 120. As another example, the processing device 140 may obtain user instructions from the terminal(s) 130 via the network 120. In some embodiments, the network 120 may include one or more network access points.

The terminal(s) 130 may include a processing unit, a display unit, a sensing unit, an input/output (I/O) unit, a storage unit, etc. The sensing unit may include a light sensor, a distance sensor, an accelerometer, a gyroscope sensor, an acoustic detector, or the like, or any combination thereof. The terminal(s) 130 may include a mobile device 130-1, a tablet computer 130-2, a laptop computer 130-3, a desktop computer 130-4, or the like, or any combination thereof. In some embodiments, the mobile device 130-1 may include a smart home device, a wearable device, a mobile device, a virtual reality device, an augmented reality device, or the like, or any combination thereof. In some embodiments, the terminal(s) 130 may be part of the processing device 140.

The processing device 140 may process data and/or information obtained from one or more components (the medical device 110, the terminal(s) 130, and/or the storage device 150) of the imaging system 100. For example, the processing device 140 may determine, based on the subject to be scanned, one or more scanning parameters of the medical device 110. As another example, the processing device 140 may obtain the image data (e.g., the PET data and the MR data) collected by the medical device 110 in a scan of the subject under the one or more scanning parameters. The PET data may be collected by the PET detector module of the medical device 110, and the MR data may be collected by the RF coil of the medical device 110. As still another example, the processing device 140 may generate, based on the PET data and the MR data, a fused image of the subject. As yet another example, the processing device 140 may determine, based on the fused image, a position of a lesion of the subject. As yet another example, the processing device 140 may transmit the fused image and/or the position of the lesion to another component (e.g., the terminal(s) 130, the storage device 150, etc.) of the imaging system 100. In some embodiments, the processing device 140 may be a single server or a server group. The server group may be centralized or distributed. In some embodiments, the processing device 140 may be local or remote. In some embodiments, the processing device 140 may be implemented on a cloud platform.

In some embodiments, the processing device 140 may be implemented by a computing device. For example, the computing device may include a processor, a storage, an input/output (I/O), and a communication port. The processor may execute computer instructions (e.g., program codes) and perform functions of the processing device 140 in accordance with the techniques described herein. The computer instructions may include, for example, routines, programs, objects, components, data structures, procedures, modules, and functions, which perform particular functions described herein. In some embodiments, the processing device 140, or a portion of the processing device 140 may be implemented by a portion of the terminal(s) 130.

The storage device 150 may store data/information obtained from the medical device 110, the terminal(s) 130, and/or any other component of the imaging system 100. In some embodiments, the storage device 150 may include a mass storage, a removable storage, a volatile read-and-write memory, a read-only memory (ROM), or the like, or any combination thereof. In some embodiments, the storage device 150 may store one or more programs and/or instructions to perform exemplary methods described in the present disclosure.

In some embodiments, the storage device 150 may be connected to the network 120 to communicate with one or more other components in the imaging system 100 (e.g., the processing device 140, the terminal(s) 130, etc.). One or more components in the imaging system 100 may access the data or instructions stored in the storage device 150 via the network 120. In some embodiments, the storage device 150 may be directly connected to or communicate with one or more other components in the imaging system 100 (e.g., the processing device 140, the terminal(s) 130, etc.). In some embodiments, the storage device 150 may be part of the processing device 140.

In some embodiments, the imaging system 100 may include one or more additional components and/or one or more components of the imaging system 100 described above may be omitted. Additionally or alternatively, two or more components of the imaging system 100 may be integrated into a single component. A component of the imaging system 100 may be implemented on two or more sub-components.

FIG. 2A is a schematic diagram illustrating an exemplary medical device according to some embodiments of the present disclosure. A medical device 200 may be an embodiment of the medical device 110 described in FIG. 1.

As shown in FIG. 2A, the medical device 200 may be represented in a 3D coordinate system (e.g., an orthogonal coordinate system) including an X-axis, a Y-axis, and the Z axis. The X-axis and the Z-axis may be in a horizontal plane, and the Y axis may be along a vertical direction. From a direction facing a front portion of the medical device 200, a direction from a right side to a left side of the medical device 200 may be designated as a positive direction of the X axis. A direction from a lower portion to an upper portion of the medical device 200 may be designated as a positive direction of the Y axis. A direction that the scanning table 204 is moved out of the scanning tunnel 210 may be designated as a positive direction of the Z axis.

As shown in FIG. 2A, the medical device 200 may include a housing 202, a scanning table 204, a PET imaging component 206, and an MRI component 208.

The housing 202 may form a scanning tunnel 210 for accommodating a subject to be scanned. In some embodiments, a shape of the scanning tunnel 210 of the housing 202 may be a circle, an oval, a square, a rectangle, etc. In some embodiments, the housing 202 may support one or more parts of the medical device 200, for example, the PET imaging component 206, the MRI component 208, etc. For example, the housing 202 may include one or more supporting frames for accommodating and supporting the PET imaging component 206 and/or the MRI component 208. Merely by way of example, FIG. 2B illustrates an exemplary housing 202 according to some embodiments of the present disclosure. As shown in FIG. 2B, the housing 202 includes a first supporting frame 262 and a second supporting frame 264, wherein the PET imaging component 206 (not shown in FIG. 2B) may be accommodated in the first supporting frame 262, and the MRI component 208 (not shown in FIG. 2B) may be accommodated in the second supporting frame 264.

The scanning table 204 may support the subject to be scanned and adjust the position of the subject in the scanning tunnel 210. In some embodiments, the subject to be scanned may lie on the scanning table 204. The scanning table 204 may be moved along a direction parallel to the Z axis as shown in FIG. 2A.

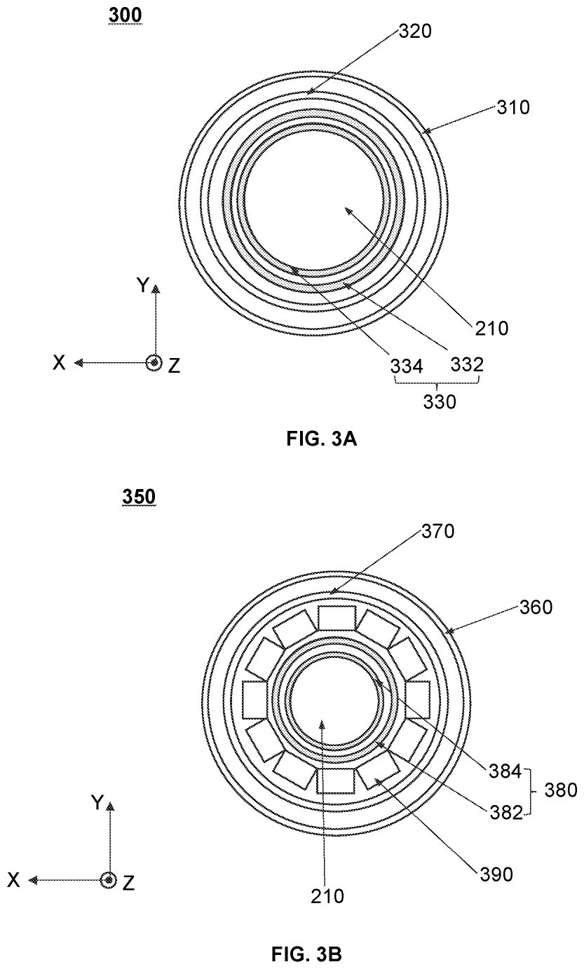

The PET imaging component 206 may be arranged along a circumference of the scanning tunnel 210. The PET imaging component 206 may be configured to detect radiation photons (e.g., gamma photons) emitted from the subject during a scan. In some embodiments, the PET imaging component 206 may include a PET detector module. The PET detector module may include a plurality of detection units arranged in a circular array around the Z axis. Each of the plurality of detection units may include a scintillation crystal and a photovoltaic sensor. The scintillation crystal may receive gamma photons. The photovoltaic sensor may include a photomultiplier tube (PMT), a silicon photomultiplier (SiPM), etc. In some embodiments, the PET detector module may be configured to detect 511 keV gamma rays generated by positron-electron annihilation events. For example, radiation photons (e.g., gamma photons) may generate fluorescence after energy deposition in the scintillation crystal, the fluorescence may be converted to electrical signals using the photovoltaic sensor, and the energy of the radiation photons may be indirectly determined based on the electrical signals. In some embodiments, the processing device 140 may process PET data related to the gamma rays, and generate a PET image by using a reconstruction algorithm. Exemplary reconstruction algorithms may include a maximum likelihood estimation algorithm, an expectation maximization algorithm, a filtered back-projection algorithm, an iterative reconstruction algorithm, or the like, or any combination thereof. More descriptions regarding a structure of a PET imaging component may be found elsewhere in the present disclosure. See, e.g., FIG. 3B, and relevant descriptions thereof.

The MRI component 208 may be arranged along the circumference of the scanning tunnel 210. In some embodiments, the MRI component 208 may be configured to detect MR signals related to the subject during the scan. In some embodiments, the MRI component 208 may include a main magnet, a gradient coil, and an RF coil. The main magnet, the gradient coil, and the RF coil may be disposed coaxially around an axial direction (i.e., the Z axis) of the scanning tunnel 210.

Merely by way of example, referring to FIG. 3A, FIG. 3A is a schematic diagram illustrating a plan view of an exemplary MRI component according to some embodiments of the present disclosure. An MRI component 300 may be an embodiment of the MRI component 208 described in FIG. 2A. An X axis, a Y axis, and a Z axis in FIG. 3A may correspond to the X axis, the Y axis, and the Z axis in FIG. 2A, respectively. As shown in FIG. 3A, the MRI component 300 may include a main magnet 310, a gradient coil 320, and an RF coil 330. The main magnet 310, the gradient coil 320, and the RF coil 330 may be disposed coaxially around a Z axis of the scanning tunnel 210.

In some embodiments, the main magnet 310 may be configured to generate a first magnetic field (also referred to as a main magnetic field). The first magnetic field may be applied to the subject to be scanned in the scanning tunnel 210. The main magnet 310 may include a permanent magnet, a superconducting electromagnet, a resistive electromagnet, etc.

The gradient coil 320 may be configured to generate a second magnetic field (also referred to as a gradient magnetic field). The gradient coil 320 may include X-gradient coils, Y-gradient coils, and Z-gradient coils. The gradient coil 320 may generate one or more magnetic field gradient pulses to the main magnetic field in the X axis, the Y axis, and the Z axis to encode spatial information of the subject. For example, the second magnetic field may be superimposed on the main magnetic field generated by the main magnet 310 so that a magnetic orientation of each proton of the subject can vary with a position of the proton within the gradient magnetic field, thereby encoding the spatial information of the subject in MR signals. In some embodiments, the X-gradient coils may be configured to generate a gradient magnetic field Gx along the X axis, the Y-gradient coils may be configured to generate a gradient magnetic field Gy along the Y axis, and the Z-gradient coils may be configured to generate a gradient magnetic field Gz along the Z axis. Therefore, the X-gradient coils, the Y-gradient coils, and the Z-gradient coils may generate three gradient magnetic fields for encoding the spatial information of the subject. In some embodiments, a radius of the gradient coil 320 may be less than a radius of the main magnet 310. For example, the gradient coil 320 may be surrounded by the main magnet 310.

The RF coil 330 may be used as RF transmitting coils and/or RF receiving coils. In some embodiments, a radius of the RF coil 330 may be less than the radius of the main magnet 310 and/or the radius of the gradient coil 320. For example, the RF coil 330 may be surrounded by the main magnet 310 and the gradient coil 320.

When the RF coil 330 is used as the RF transmitting coil(s), the RF coil 330 may emit RF signals (e.g., RF pulses), and the RF signals may provide a third magnetic field. The third magnetic field may be used to excite the MR signals related to the subject. In some embodiments, the third magnetic field may be perpendicular to the first magnetic field. When the RF coil 330 is used as the RF receiving coil(s), the RF coil 330 may be used to detect the MR signals (e.g., echo signals) from the subject.

In some embodiments, the MRI component 300 may include a receiving amplifier and an analog to digital converter (ADC). The receiving amplifier may receive the MR signals from the RF coil 330, and amplify the MR signals. Further, the receiving amplifier may transmit the amplified MR signals to the ADC. The ADC may be configured to convert the MR signals from analog signals to digital signals. Accordingly, the digital MR signals may be used to generate an MR image of the subject. For example, the digital MR signals may be transmitted to the processing device 140, and the processing device 140 may process the digital MR signals to generate an MR image of the subject.

In some embodiments, the RF coil 330 of the MRI component 208 may include a first RF coil and a second RF coil. The first RF coil and the second RF coil may be disposed coaxially around the axial direction (i.e., the Z axis) of the scanning tunnel 210. A projection of the second RF coil along a radial direction of the scanning tunnel 210 may cover at least a portion of a gap of the first RF coil. As used herein, a gap of the first RF coil refers to an interspace between components of the first RF coil, e.g., between windings of a helical RF coil, parallel units of a birdcage coil, coil units of a phased array coil; a projection of the second RF coil along a radial direction of the scanning tunnel 210 refers to a projection of the second RF coil along the radial direction of the scanning tunnel 210 on the surface or plane where the first RF coil is located.

Merely by way of example, as shown in FIG. 3A, the RF coil 330 may include a first RF coil 332 and a second RF coil 334. The first RF coil 332 and the second RF coil 334 may be disposed coaxially around the axial direction (i.e., the Z axis) of the scanning tunnel 210. For example, a radius of the second RF coil 334 may be less than a radius of the first RF coil 332, and the first RF coil 332 may be arranged along a periphery of the second RF coil 334.

In some embodiments, a portion of a projection of the second RF coil 334 along the radial direction of the scanning tunnel 210 may cover at least a portion of a gap of the first RF coil 332. For example, the projection of the second RF coil 334 along the radial direction of the scanning tunnel 210 may cover at least a portion of the gap of the first RF coil 332, and the projection of the second RF coil 334 may not cover the first RF coil 332. In other words, the first RF coil 332 and the second RF coil 334 may be interleaved and not overlapped when looking the first and second RF coils inside the scanning tunnel 210. As another example, the projection of the second RF coil 334 along the radial direction of the scanning tunnel 210 may cover at least a portion of the gap of the first RF coil 332, and the projection of the second RF coil 334 may cover a portion of the first RF coil 332. In other words, the first RF coil 332 and the second RF coil 334 may be interleaved and overlapped when looking the first and second RF coils inside the scanning tunnel 210. In some cases, the first RF coil 332 may be inserted into the second RF coil 334. Alternatively, the second RF coil 334 may be inserted into the first RF coil 332.

In some embodiments, the second RF coil 334 may be detachably mounted in the medical device 300. For example, the medical device 300 may include one or more positioning components configured to fix the second RF coil 334 on an inner surface of a housing (e.g., the housing 202). The second RF coil 334 may be detachably mounted in the medical device 300 through the one or more positioning components. In some embodiments, the medical device 300 may include a plurality of candidate second RF coils with different coil parameters, and a target second RF coil (i.e., the second RF coil 334) may be selected from the plurality of candidate second RF coils based on a scanning condition of the subject. Exemplary coil parameters may include a location, a size, a thickness, a type, or the like, or any combination thereof. The scanning condition may relate to, for example, a region of interest (ROI) of the subject, a required signal-to-noise ratio of MR imaging or PET imaging, a required image quality, etc. For example, when an ROI is the chest of the subject, a candidate second RF coil corresponding to the chest may be determined as the target second RF coil (i.e., the second RF coil 334). As another example, if the signal-to-noise ratio of the MR imaging needs to be large, a candidate second RF coil with a largest size may be determined as the target second RF coil. As still another example, if the signal-to-noise ratio of the PET imaging needs to be large, a candidate second RF coil with a least thickness may be determined as the target second RF coil. In this way, different second RF coils may be used to adapt to different scanning conditions. In addition, a detachable second RF coil can be easily assembled without changing the structure of the medical device, and widely used in existing medical devices (e.g., PET-MRI devices, MRI devices, etc.).

In some embodiments, the first RF coil 332 and the second RF coil 334 may be integrated in the medical device 300. For example, when the medical device 300 is produced, the first RF coil 332 and the second RF coil 334 may be integrated in the medical device 300. Therefore, the second RF coil 334 does not need to be mounted before the scan, which can simplify the scanning process, and improve the efficiency of the scan.

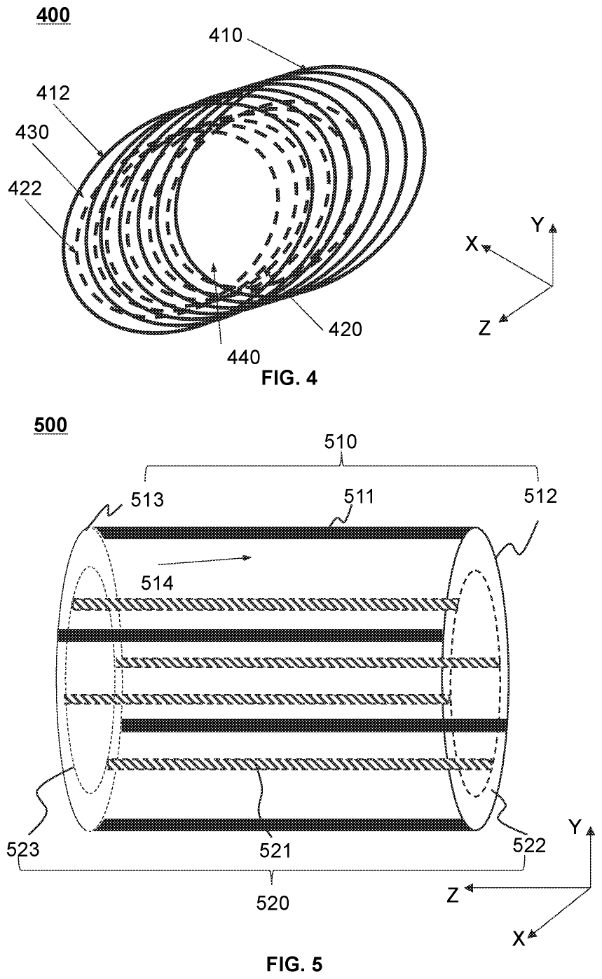

In some embodiments, a type of the first RF coil 332 or the second RF coil 334 may include a full volume coil, a body coil, an orthogonal coil (e.g., a birdcage coil), a linearity coil (e.g., a helical coil), a phased array coil, etc. The linearity coil may receive MR signals from a direction perpendicular to the first magnetic field. The orthogonal coil may receive the MR signals from two directions perpendicular to the first magnetic field. The phased array coil may include a plurality of coil units, and receive the MR signals from a plurality of directions. In some embodiments, the first RF coil 332 and the second RF coil 334 may include a single-channel coil or a multi-channel coil (e.g., a dual-channel coil, a four-channel coil, an eight-channel coil, a sixteen-channel coil, a thirty-two-channel coil, etc.) In some embodiments, the type of the first RF coil 332 may be the same as the type of the second RF coil 334. For example, both the first RF coil 332 and the second RF coil 334 may be orthogonal coils, helical coils, or phased array coils. More descriptions regarding an arrangement of the first and second RF coils when they have the same type may be found elsewhere in the present disclosure. See, e.g., FIGS. 4-6B, and relevant descriptions thereof.

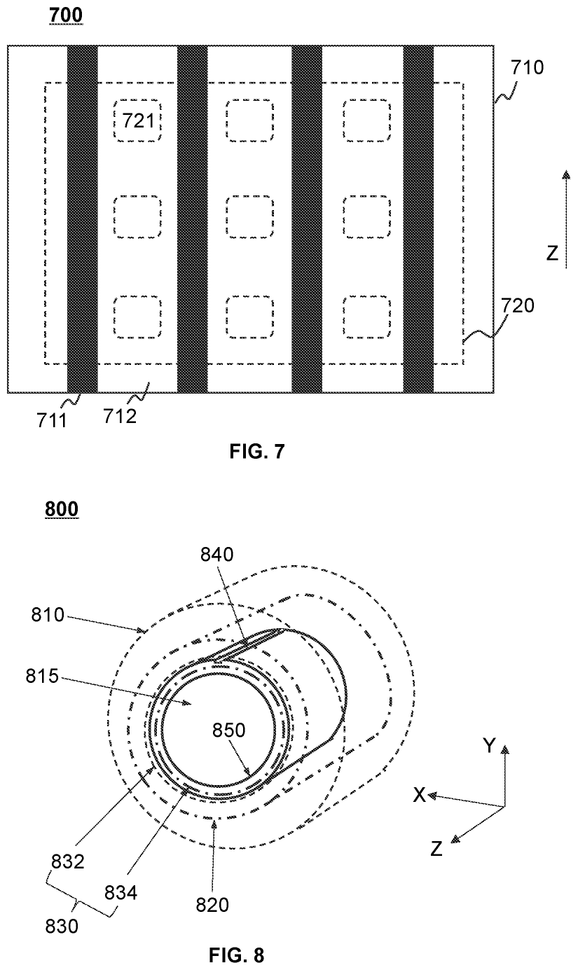

In some embodiments, the type of the first RF coil 332 may be different from the type of the second RF coil 334. For example, the first RF coil 332 may be the orthogonal coil, and the second RF coil 334 may be the phased array coil. As another example, the first RF coil 332 may be the phased array coil, and the second RF coil 334 may be the orthogonal coil. More descriptions regarding an arrangement of the first and second RF coils when they have different types may be found elsewhere in the present disclosure. See, e.g., FIG. 7 and relevant descriptions thereof.

In some embodiments, a count of the first RF coil 332 may be one or multiple. For example, the MRI component 300 may include a plurality of first RF coils 332. The plurality of first RF coils 332 may be disposed coaxially around the axial direction of the scanning tunnel 210 like multiple staked layers, or disposed in parallel along the Z axis. Similarly, a count of the second RF coil 334 may be one or multiple. For example, the MRI component 300 may include a plurality of second RF coils 334. The plurality of second RF coils 334 may be disposed coaxially around the axial direction of the scanning tunnel 210 like multiple staked layers, or disposed in parallel along the Z axis. In some embodiments, the plurality of first RF coils 332 may be arranged along a circumference of the plurality of second RF coils 334.

In some embodiments, the PET imaging component 206 and the MRI component 208 may form an embedded structure. Merely by way of example, referring to FIG. 3B, FIG. 3B is a schematic diagram illustrating a planned view of an exemplary medical device according to some embodiments of the present disclosure. A medical device 350 may be an embodiment of the medical device 200 described in FIG. 2A. An X axis, a Y axis, and a Z axis in FIG. 3B may correspond to the X axis, the Y axis, and the Z axis in FIG. 2A, respectively.

As shown in FIG. 3B, the medical device 350 may include an MRI component (including a main magnet 360, a gradient coil 370, an RF coil 380) and a PET imaging component (including a PET detector module 390). The RF coil 380 may include a first RF coil 382 and a second RF coil 384. The main magnet 360, the gradient coil 370, and the RF coil 380 may be similar to the main magnet 310, the gradient coil 320, and the RF coil 330, respectively. The PET detector module 390 may be similar to the PET detector module in the PET imaging component 206. The main magnet 360, the gradient coil 370, the RF coil 380, and the PET detector module 390 may be disposed coaxially around a Z axis of the scanning tunnel 210, and the PET detector module 390 may be disposed between the gradient coil 370 and the RF coil 380. That is, the MRI component and the PET imaging component in the medical device 350 may form an embedded structure.

In some embodiments, the second RF coil 384 may further include a preamplifier. The preamplifier may be configured to amplify MR signals received by the second RF coil 384. For example, the preamplifier may be disposed outside a field of view (FOV) of the PET imaging component (or the PET detector module 390).

In some embodiments, the PET imaging component 206 and the MRI component 208 may form a tandem structure. For example, the PET imaging component 206 may be detachably or non-detachably arranged in series adjacent to the MRI component 208 along the Z axis. Merely by way of example, as shown in FIG. 2B, the PET imaging component may be disposed in the first supporting frame 262 of the housing 202, and the MRI component may be disposed in the second supporting frame 264 of the housing 202. The second supporting frame 264 may be located in front of the first supporting frame 262 along the Z axis. That is, the PET imaging component may be located in front of the MRI component along the Z axis. As another example, the PET imaging component may be located in rear of the MRI component along the Z axis.

By introducing the second RF coil, the medical device may acquire more MR signals related to the subject than a medical device that only includes the first RF coil, which can improve a signal-to-noise ratio and the imaging quality of MR imaging. Further, the first RF coil and the second RF coil can acquire MR signals, respectively. Two independent and parallel MR images can be generated, which can further improve the signal-to-noise ratio of MR imaging. Since the signal-to-noise ratio of MR imaging is improved, a thickness of the first RF coil and/or the second RF coil can be reduced, so that attenuations of the radiation photons emitted from the subject may be reduced, thereby improving the imaging quality of PET imaging. Therefore, the imaging quality of the medical device may be improved, and the accuracy and the image quality of a fused image generated based on the radiation photons and the MR signals may be improved, which, in turn, improves the precision of subsequent diagenesis and treatment planning performed based on the fused image.

FIG. 4 is a schematic diagram illustrating an exemplary RF coil according to some embodiments of the present disclosure.

An RF coil 400 may be an embodiment of the RF coil 330 described in FIG. 3A. An X axis, a Y axis, and a Z axis in FIG. 4 may correspond to the X axis, the Y axis, and the Z axis in FIG. 3A, respectively.

As shown in FIG. 4, the RF coil 400 may include a first RF coil 410 and a second RF coil 420. In some embodiments, the first RF coil 410 may include a linearity coil. For example, the first RF coil 410 may be a helical RF coil donated by solid lines in FIG. 4. That is, the first RF coil 410 may have a helical arrangement formed by multiple windings (e.g., a winding 412) around the Z axis. Gaps 430 may be formed between the multiple windings. In some embodiments, the second RF coil 420 may also include a linearity coil. For example, the second RF coil 420 may be a helical RF coil donated by dotted lines in FIG. 4. That is, the second RF coil 420 may have a helical arrangement formed by multiple windings (e.g., a winding 422) around the Z axis.

In some embodiments, a projection of the windings of the second RF coil 420 along the radial direction of a scanning tunnel 440 may cover at least a portion of the gaps 430 formed by the windings of the first RF coil 410. For example, as shown in FIG. 4, the first RF coil 410 and the second RF coil 420 are interleaved when looking the first and second RF coils inside the scanning tunnel, and a projection of each winding 422 of the second RF coil 420 along the radial direction of the scanning tunnel 440 may be located in one gap 430 formed by adjacent windings of the first RF coil 410.

FIG. 5 is a schematic diagram illustrating an exemplary RF coil according to some embodiments of the present disclosure.

An RF coil 500 may be an embodiment of the RF coil 330 described in FIG. 3A. An X axis, a Y axis, and a Z axis in FIG. 5 may correspond to the X axis, the Y axis, and the Z axis in FIG. 3A, respectively.

As shown in FIG. 5, the RF coil 500 may include a first RF coil and a second RF coil. In some embodiments, the first RF coil may include an orthogonal coil, for example, a birdcage coil 510 as shown in FIG. 5. In some embodiments, the second RF coil may include an orthogonal coil, for example, a birdcage coil 520 as shown in FIG. 5. In some embodiments, gaps 514 may be formed between the plurality of parallel units 511 of the first RF coil (i.e., the birdcage coil 510).

In some embodiments, a birdcage coil may include a plurality of parallel units (also referred to as leg portions) extend along the Z axis and circular units (also referred to as end portions) that are located at the ends of the birdcage coil and are used to connect the plurality of parallel units. For example, the birdcage coil 510 includes parallel units 511 (denoted as solid lines in FIG. 5) and circular units 512 and 513 (denoted as solid circles in FIG. 5); the birdcage coil 520 includes parallel units 521 (denoted as dotted lines in FIG. 5) and circular units 522 and 523 (denoted as dotted circles in FIG. 5). In some embodiments, a parallel unit may be a metal conductor. For example, the parallel unit may be a metal plate, such as, a copper plate, an aluminum plate, etc. As another example, a parallel unit may be a metal hollow tube, such as, a copper tube, an aluminum tube, etc. In some embodiments, a parallel unit may include a slot extending along the Z axis, which can reduce an eddy current effect in the metal conductor. In some embodiments, a circular unit may be a metal ring, such as, a copper ring, an aluminum ring, etc. In some embodiments, a circular unit may be disposed with one or more capacitive elements.

In some embodiments, a projection of the parallel units 521 of the birdcage coil 520 along a radial direction of a scanning tunnel may cover at least a portion of the gaps 514 formed by the plurality of parallel units 511 of the birdcage coil 510. For example, as shown in FIG. 5, a projection of each of the plurality of parallel unit 521 of the birdcage coil 520 along the radial direction of the scanning tunnel may be located at one gap 514 formed by adjacent parallel units 511 of the birdcage coil 510. As another example, the parallel units 511 of the birdcage coil 510 and the parallel units 521 of the birdcage coil 520 may be disposed along different radial directions. Therefore, a projection of at least one of the parallel units 521 of the birdcage coil 520 along the radial direction may cover at least a portion of a gap/interspace of two adjacent parallel units 511 of the birdcage coil 510. In other word, at least one of the parallel units 521 of the birdcage coil 520 may be not aligned with any of the parallel units 511 of the birdcage coil 510 along the radial direction of the scanning tunnel. Therefore, a projection of at least one of the parallel units 521 of the birdcage coil 520 along the radial direction of the scanning tunnel may cover at least a portion of a gap/interspace of two adjacent parallel units 511 of the birdcage coil 510. As still another example, two adjacent parallel units 511 of the birdcage coil 510 and a central axis of the birdcage coil 510 may form an interspace, and at least one parallel unit 521 of the birdcage coil 520 may be disposed in the interspace. Accordingly, a projection of the at least one parallel unit 521 of the birdcage coil 520 may be located in the interspace, and a projection of the at least one parallel unit 521 of the birdcage coil 520 along the radial direction may cover at least a portion of a gap of the two adjacent parallel units 511 of the birdcage coil 510.

In some embodiments, a portion of the circular units of the birdcage coil 510 and a portion of the circular units of the birdcage coil 520 may be disposed on a same position along an axial direction of the scanning tunnel. For example, as shown in FIG. 5, the circular unit 512 of the birdcage coil 510 and the circular unit 522 of the birdcage coil 520 may be disposed on a same position along the axial direction of the scanning tunnel, and the circular unit 513 of the birdcage coil 510 and the circular unit 523 of the birdcage coil 520 may be disposed on a same position along the axial direction of the scanning tunnel. That is, the circular unit 512 and the circular unit 522 may be concentric circles, and the circular unit 513 and the circular unit 523 may be concentric circles. In some embodiments, a portion of the circular units of the birdcage coil 510 and a portion of the circular units of the birdcage coil 520 may be disposed on different positions along the axial direction of the scanning tunnel. That is, the circular units of the birdcage coil 510 and the circular units of the birdcage coil 520 may be disposed in a dislocation distribution. For example, the circular unit 512 of the birdcage coil 510 and the circular unit 522 of the birdcage coil 520 may be disposed on different positions along the axial direction of the scanning tunnel, and/or the circular unit 513 of the birdcage coil 510 and the circular unit 523 of the birdcage coil 520 may be disposed on different positions along the axial direction of the scanning tunnel. That is, the circular unit 512 and the circular unit 522 may not be concentric circles, and the circular unit 513 and the circular unit 523 may not be concentric circles.

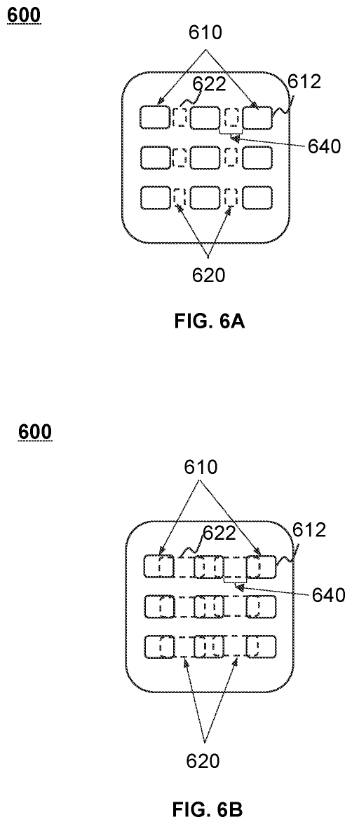

FIGS. 6A and 6B are schematic diagrams illustrating an exemplary RF coil according to some embodiments of the present disclosure.

An RF coil 600 may be an embodiment of the RF coil 330 described in FIG. 3A. As shown in FIGS. 6A and 6B, the RF coil 600 may include a phased array coil 610 (i.e., a first RF coil) and a phased array coil 620 (i.e., a second RF coil). The phased array coil 610 may include a plurality of coil units 612 arranged in an array. Similarly, the phased array coil 620 may include a plurality of coil units 622 arranged in an array. Gaps 640 may be formed between adjacent coil units 612 of the phased array coil 610.

In some embodiments, the phased array coil 610 and the phased array coil 620 may be disposed coaxially around the Z axis. For example, a radius of the phased array coil 620 may be less than a radius of the phased array coil 610, and the phased array coil 610 may be arranged along a circumference of the phased array coil 620. In some embodiments, a projection of the phased array coil 620 along a radial direction of the scanning tunnel may cover at least a portion of the gaps 640 of the phased array coil 610. For example, as shown in FIG. 6A, a projection of each coil unit 622 of the phased array coil 620 along the radial direction of the scanning tunnel may cover a portion of a gap 640 formed by adjacent coil units 612 of the phased array coil 610, and the projection of each coil unit 622 may not cover any coil unit 612. As another example, as shown in FIG. 6B, a projection of each coil unit 622 of the phased array coil 620 along the radial direction of the scanning tunnel covers a portion of a gap 640 formed by adjacent coil units 612 of the phased array coil 610, and the projection of each coil unit 622 may also cover a portion of the plurality of coil units 612. In some embodiments, an overlapped area between the projection of the coil units 622 and the coil units 612 may be less than an area threshold, such as, 1%, 2%, 5%, 10%, 20%, 30%, 40%, 50%, etc., of an area of the plurality of coil units 622 or the plurality of coil units 612.

FIG. 7 is a schematic diagram illustrating an exemplary RF coil according to some embodiments of the present disclosure.

An RF coil 700 may be an embodiment of the RF coil 330 described in FIG. 3A. A Z axis in FIG. 7 may correspond to the Z axis in FIG. 3A.

As shown in FIG. 7, the RF coil 700 may include a first RF coil (i.e., a birdcage coil 710) and a second RF coil (i.e., a phased array coil 720). In some embodiments, the birdcage coil 710 may include a plurality of parallel units 711 extending along the Z axis. Gaps 712 may be formed between the plurality of parallel units 711. The phased array coil 720 may include a plurality of coil units 721 arranged in an array.

In some embodiments, a projection of the coil units 721 of the phased array coil 720 along a radial direction of a scanning tunnel may cover at least one a portion of the gaps 712 of the birdcage coil 710. For example, as shown in FIG. 7, the phased array coil 720 may include a plurality of columns of coil units 721 arranged along the Z axis, and a projection of each column of coil units 721 along the radial direction may be located in the one gap 712 formed by two adjacent parallel units 711 of the birdcage coil 710. In other words, at least one of the coil units 721 of the phased array coil 720 may not be aligned with the parallel units 711 of the birdcage coil 710 along a radial direction. Therefore, a projection of the at least one of the coil units 721 along the radial direction may be located in the one gap 712 formed by two adjacent parallel units 711 of the birdcage coil 710. The gap 712 may referred to an interspace between adjacent parallel units 711. As another example, the parallel units 711 of the birdcage coil 710 and the coil units 721 of the phased array coil 720 may be disposed along different radial directions. Therefore, a projection of at least one of the coil units 721 of the phased array coil 720 along the radial direction may cover at least a portion of a gap/interspace of two adjacent parallel units 711 of the birdcage coil 710. As still another example, two adjacent parallel units 711 of the birdcage coil 710 and a central axis of the birdcage coil 710 may form an interspace, and at least one coil unit 721 of the phased array coil 720 may be disposed in the interspace. Accordingly, a projection of the at least one coil unit 721 of the phased array coil 720 may be located in the interspace, and a projection of the at least one coil unit 721 of the phased array coil 720 along the radial direction may cover at least a portion of a gap of the two adjacent parallel units 711 of the birdcage coil 710.

FIG. 8 is a schematic diagram illustrating an exemplary medical device according to some embodiments of the present disclosure.

A medical device 800 may be an embodiment of the medical device 200 described in FIG. 2A. An X axis, a Y axis, and a Z axis in FIG. 8 may correspond to the X axis, the Y axis, and the Z axis in FIG. 2A, respectively.

As shown in FIG. 8, the medical device 800 may include a housing 810, a PET imaging component 820, and an RF coil 830. The RF coil 830 may include a first RF coil 832 and a second RF coil 834.

The housing 810 may form a scanning tunnel 815 for accommodating a subject to be scanned. The first RF coil 832 may be disposed inside the housing 810, and the second RF coil 834 may be disposed on an inner surface of the housing 810. The first RF coil 832 and the second RF coil 834 may be disposed coaxially around an axial direction of the scanning tunnel 815. For example, the housing 810 may include a first wall and a second wall. The second wall may form the scanning tunnel 815. The first RF coil 832 may be disposed between the first wall and the second wall, and the second RF coil 834 may be located on an inner surface of the second wall.

In some embodiments, the second RF coil 834 may be detachably mounted in the medical device 800. As shown in FIG. 8, the medical device 800 may further include one or more positioning components 840. The one or more positioning components 840 may be configured to fix the second RF coil 834 on the inner surface of the housing 810 (e.g., the second wall of the housing 810), so that the second RF coil 834 can be accurately inserted into a corresponding position of the medical device 800. In some embodiments, a positioning component 840 may include a positioning groove that matches a positioning block (not shown in FIG. 8) on the inner surface of the housing 810. The one or more positioning components 840 may have a simple structure and have good positioning and fixing effects, so that the stability of the medical device 800 can be improved.

In some embodiments, the one or more positioning components 840 may include a positioning block that matches a positioning groove on the inner surface of the housing 810, which can also fix the second RF coil 834, and improve the stability of the medical device 800. A connection between the second RF coil 834 and the medical device 800 may include other detachable connections, such as, a screw connection, a pin connection, a key connection, a buckle connection, a magnetic attraction connection, or the like, or any combination thereof. In some embodiments, at least one of the one or more positioning components 840 may include an interface (not shown in FIG. 8) used for current conduction and signal transmission of the second RF coil 834. In some embodiments, the second RF coil 834 may include a plurality of sub-coils, and each of the plurality of sub-coils may correspond to an interface. Accordingly, a portion of the plurality of sub-coils that is used to receive MR signals may be determined by selecting interface(s) corresponding to the portion of the plurality of sub-coils.

In some embodiments, the one or more positioning components 840 and matching component(s) (e.g., the positioning groove, the positioning block, etc.) on the inner surface of the housing 810 may be connected in different manners to achieve different relative positions between the first RF coil 832 and the second RF coil 834. For example, the one or more positioning components 840 may be inserted into a matching component on the inner surface of the housing 810 (not shown in FIG. 8). Alternatively, the one or more positioning components 840 may be inserted into a matching component at an angle to the inner surface of the housing 810. Therefore, relative positions between the first RF coil 832 and the second RF coil 834 may be different.

In some embodiments, the second RF coil 834 may be pulled out of or inserted into the scanning tunnel 815 from a front end (one end near a scanning bed along the Z axis) or a rear end (one end away from the scanning bed along the Z axis) of the RF coil 830. Therefore, the second RF coil 834 may be separated from or mounted on the medical device 800.

In some embodiments, the medical device 800 may further include a second housing 850. That is, the housing of the medical device 800 may include two separate housing, i.e., a first housing (i.e., the housing 810) and the second housing 850. The first housing (i.e., the housing 810) and the second housing 850 may be disposed coaxially around the axial direction of the scanning tunnel 815. The second housing 850 may form the scanning tunnel 815. The first RF coil 832 may be disposed inside the first housing 810, and the second RF coil 834 may be disposed inside the second housing 850. The second housing 850 may be configured to protect the second RF coil 834 and prevent the subject from contacting with the second RF coil 834. In some embodiments, the second housing 850 may be detachably mounted in the medical device 800. For example, the second housing 850 may be mounted in the medical device 800 through a detachable connection, such as, a screw connection, a pin connection, a key connection, a buckle connection, a magnetic attraction connection, or the like, or any combination thereof. In some embodiments, the second housing 850 may be non-detachably mounted in the medical device 800. Accordingly, the second RF coil 834 may be pulled out of or inserted in the scanning tunnel 815 from the front end or the rear end of the RF coil 830.

In some embodiments, the first RF coil 832 and the second RF coil 834 may be integrated in the medical device 800. For example, the first RF coil 832 and the second RF coil 834 may be mounted inside the housing 810. In some embodiments, the first RF coil 832 and the second RF coil 834 may be integrally formed. In some embodiments, the first RF coil 832 and the second RF coil 834 may be integrally formed on the medical device 800. By using the integrated structure, no RF coil needs to be mounted on the medical device 800 during the scan, which can simplify the scanning process, save the scanning time, and improve the efficiency of the medical device 800.

Since the medical device 800 includes the PET detector module 820 and the RF coil 830, the PET detector module 820 and the RF coil 830 may interact with each other, which may reduce the imaging quality of the medical device 800. For example, referring back to FIG. 3B, the PET detector module 390 may be disposed between the gradient coil 370 and the RF coil 380. Therefore, the RF coil 380 may attenuate the radiation photons, which can reduce a signal-to-noise ratio of photon signals detected by the PET detector module 390 of the medical device 350 and reduce the imaging quality of the PET imaging component of the medical device 350.

In some embodiments, a high-density component in the first RF coil 832 and the second RF coil 834 of the RF coil 830 may be disposed outside a field of view (FOV) of the PET detector module 820. For example, the second RF coil 834 may include a preamplifier, and the preamplifier may be disposed outside an FOV of the PET detector module 820. In some embodiments, a radial thickness of the first RF coil 832 and/or the second RF coil 834 may be reduced, and a circumferential width of the first RF coil 832 and/or the second RF coil 834 may be increased. Therefore, while reducing the attenuation of the radiation photons in the radial direction, a cross-sectional area of the first RF coil 832 and/or the second RF coil 834 may remain unchanged, which can ensure the imaging quality of the MRI component. By disposing the high-density component outside the FOV of the PET detector module 820 and/or reducing the radial thickness of the first RF coil 832 and/or the second RF coil 834, the attenuation of the radiation photons in the radial direction may be reduced, which can improve the imaging quality of the PET component. In addition, by increasing the circumferential width of the first RF coil 832 and/or the second RF coil 834, gaps between the first RF coil 832 and/or gaps the second RF coil 834 may be reduced, which can improve the uniformity of the attenuation of the radiation photons, thereby reducing the difficulty of attenuation correction and improving the accuracy of the PET imaging.

It should be noted that the medical devices 200, 300, 350, and 800 and the RF coil 400, 500, 600, and 700 are provided for illustration purposes, and are not intended to limit the scope of the present disclosure. For persons having ordinary skills in the art, multiple variations and modifications may be made under the teachings of the present disclosure. However, those variations and modifications do not depart from the scope of the present disclosure. For example, the position, the shape, and/or the size of a component shown in figures can be modified according to actual needs. As another example, one or more additional components may be added, and/or one or more components described above may be omitted.

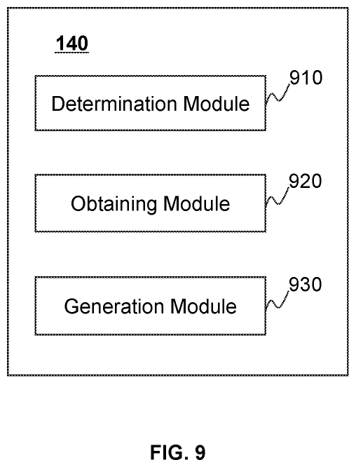

FIG. 9 is a block diagram illustrating an exemplary processing device 140 according to some embodiments of the present disclosure. In some embodiments, the modules illustrated in FIG. 9 may be implemented on the processing device 140. In some embodiments, the processing device 140 may be in communication with a computer-readable storage medium (e.g., the storage device 150 illustrated in FIG. 1) and may execute instructions stored in the computer-readable storage medium. The processing device 140 may include a determination module 910, an obtaining module 920, and a generation module 930.

The determination module 910 may be configured to determine, based on a subject to be scanned, one or more scanning parameters of a medical device. The medical device may include a housing, a PET detector module, and an RF coil. More descriptions regarding the determination of the one or more scanning parameters may be found elsewhere in the present disclosure. See, e.g., operation 1002 and relevant descriptions thereof.

The obtaining module 920 may be configured to obtain PET data and MR data collected by the medical device in a scan of the subject under the one or more scanning parameters. The PET data may be collected by the PET detector module, and the MR data may be collected by the RF coil. More descriptions regarding the obtaining of the PET data and the MR data may be found elsewhere in the present disclosure. See, e.g., operation 1004 and relevant descriptions thereof.

The generation module 930 may be configured to generate, based on the PET data and the MR data, a fused image of the subject. More descriptions regarding the generation of the fused image of the subject may be found elsewhere in the present disclosure. See, e.g., operation 1006 and relevant descriptions thereof.

In some embodiments, the determination module 910 may be further configured to determine, based on the fused image, a position of a lesion of the subject. More descriptions regarding the determination of the position of the lesion of the subject may be found elsewhere in the present disclosure. See, e.g., operation 1008 and relevant descriptions thereof.

It should be noted that the above descriptions of the processing device 140 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the processing device 140 may include one or more other modules. For example, the processing device 140 may include a storage module to store data generated by the modules in the processing device 140. In some embodiments, any two of the modules may be combined as a single module, and any one of the modules may be divided into two or more units.

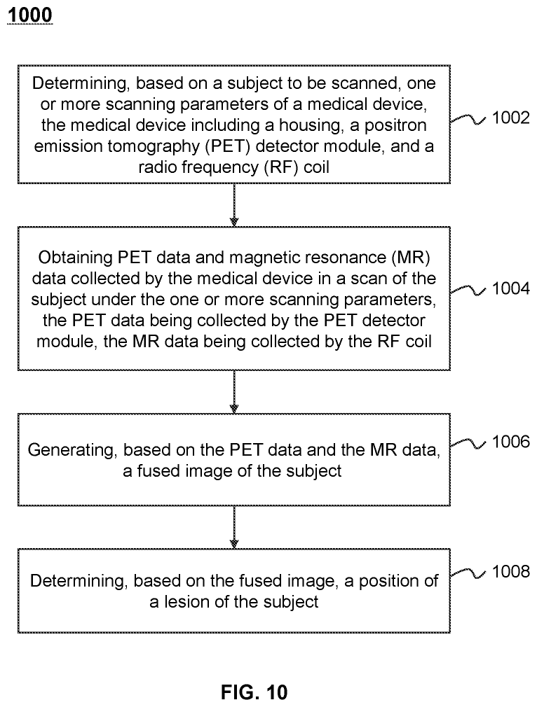

FIG. 10 is a flowchart illustrating an exemplary process 1000 for generating a fused image of a subject according to some embodiments of the present disclosure. In some embodiments, process 1000 may be implemented in the imaging system 100 illustrated in FIG. 1. For example, the process 1000 may be stored in the storage device 150 in the form of instructions (e.g., an application), and invoked and/or executed by the processing device 140. The operations of the illustrated process presented below are intended to be illustrative. In some embodiments, the process 1000 may be accomplished with one or more additional operations not described, and/or without one or more of the operations discussed. Additionally, the order in which the operations of the process 1000 as illustrated in FIG. 10 and described below is not intended to be limiting.

In some embodiments, the fused image of the subject may be captured using a medical device (e.g., the medical device 110). The medical device may be used for simulation positioning in diagnosis and/or radiotherapy. The simulation positioning before the diagnosis and/or radiotherapy may be used to determine information (e.g., a position, an area, a range, a relationship with surrounding tissue(s) and vital organ(s), etc.) of a lesion (e.g., a tumor), which can provide anatomical information for treatment planning (e.g., determining an outline of a target, a radiation dose, a radiotherapy plan, etc.).

An MR image has high soft tissue contrast and anatomical imaging accuracy. A boundary between the lesion and the surrounding tissue(s) in the MR image is relatively clear, and a range of artifact(s) generated by high atomic number material(s) is also relatively small. Therefore, the boundary between the lesion and the surrounding tissue(s) in the MR image may be clearly distinguished. In addition, MRI does not rely on ionizing radiation imaging, and multiple scans can be performed without increasing the radiation dose, which can improve the imaging quality of MRI without bringing additional damage on the subject.

A PET image can be used to determine a range of a lesion (e.g., a tumor) with metabolic activity, and determine a boundary of the lesion. Further, a measurement and evaluation of a heterogeneous distribution of standardized uptake values (SUV) in the lesion based on the PET image also provide an important basis for the biological intensity-modulated radiotherapy. At the same time, the PET image can be used to identify and evaluate the effect of radiotherapy. Therefore, a radiotherapy plan can be adjusted based on the PET image and the corresponding evaluation, which can improve the effect of a mid-term or long-term radiotherapy.

In some embodiments, the medical device may be an integrated PET-MR device. The fused image generated using the medical device may provide more information than an image generated by a single modality device (e.g., a PET device or an MR device), which can be used to determine an accurate position of a lesion of the subject, improve the accuracy of lesion staging, and provide a basis for treatment plan optimization. In some embodiments, the position of a lesion may be determined by performing the following operations.

In 1002, the processing device 140 (e.g., the determination module 910) may determine, based on the subject to be scanned, one or more scanning parameters of the medical device.

In some embodiments, the medical device may include a housing, a PET detector module, and an RF coil. More descriptions regarding the medical device may be found elsewhere in the present disclosure. See, e.g., FIGS. 1-8 and relevant descriptions thereof.

In some embodiments, the scanning parameter(s) may include a scanning range, a scanning distance, a scanning sequence, a type and/or a dose of a PET imaging agent, a scanning time, a field of view (FOV), or the like, or any combination thereof. As used herein, the scanning range refers to a region to be scanned on the subject. The scanning distance refers to a distance between the PET detector module and the subject (e.g., the scanning region of the subject) and/or a distance between the RF coil and the subject. The scanning sequence refers to a sequence (e.g., a spin echo sequence, a gradient echo sequence, a diffusion sequence, an inversion recovery sequence, etc.) applied by the MRI component of the medical device on the subject. The PET imaging agent refers to an agent that is injected into the subject for PET imaging. The type of the PET imaging agent may include a sugar metabolic imaging agent, an amino acid metabolic imaging agent, an anaerobic metabolism imaging agent, or the like, or any combination thereof. The dose of the PET imaging agent refers to a concentration or an amount of the PET imaging agent.

In some embodiments, the processing device 140 may obtain a scanning protocol of the subject, and determine the one or more scanning parameters of the medical device based on the scanning protocol of the subject. The scan protocol may include, for example, scanning parameter(s), a scanning portion of the subject, feature information of the subject (e.g., the gender, the body shape), or the like, or any combination thereof. The scanning protocol may be previously generated (e.g., manually input by a user or determined by the processing device 140) and stored in a storage device (e.g., the storage device 150). The processing device 140 may retrieve the scanning protocol from the storage device, and determine the one or more scanning parameters of the medical device based on the scanning protocol.

In some embodiments, the processing device 140 may determine a scanning posture of the subject on the medical device based on the scanning protocol of the subject. For example, the scanning protocol may include the scanning portion of the subject, and the processing device 140 may determine the scanning posture based on the scanning portion. The scanning posture may refer to a posture of the subject on the medical device during the scan. In some embodiments, the scanning posture may include a supine posture, a prone posture, a lateral posture, or any other suitable postures, such as a posture adjusted for a patient with a limb tumor.

In some embodiments, the one or more scanning parameters of the medical device may include that whether a scanning table includes a fixing component. For example, the processing device 140 may determine whether the scanning table needs a fixing component based on the scanning posture of the subject. The fixing component may be used to fix and/or position the subject. For example, the fixing component may include a fixing mold (e.g., a fixing plate, etc.), a plastic film that can be covered on a surface of the subject to fix the subject, etc.

In some embodiments, when a second RF coil is detachably mounted in the medical device, the one or more scanning parameters of the medical device may include a coil parameter of the second RF coil. The coil parameter of the second RF coil may include a location, a size, a thickness, a type, or the like, or any combination thereof, of the second RF coil. In some embodiments, the coil parameter of the second RF coil may be determined based on a scanning condition of the subject. The scanning condition may relate to, for example, a region of interest (ROI) of the subject, a required signal-to-noise ratio of MR imaging or PET imaging, a required image quality, etc. For example, when the ROI is the chest of the subject, the processing device 140 may determine a candidate second RF coil corresponding to the chest as the second RF coil. As another example, if the signal-to-noise ratio of the MR imaging needs to be large, the processing device 140 may determine a second RF coil with a largest size to be mounted in the medical device. As still another example, if the signal-to-noise ratio of the PET imaging needs to be large, the processing device 140 may determine a second RF coil with a least thickness to be mounted in the medical device. In this way, different second RF coils may be used to adapt to different scanning conditions. In some embodiments, after the second RF coil is determined based on the coil parameter and/or the scanning condition, the second RF coil may be mounted in the medical device. For example, the second RF coil may be fixed to the medical device before the subject is injected the PET imaging agent. In some embodiments, the processing device 140 may further determine whether the mounted second RF coil satisfies the coil parameter. For example, the processing device 140 may determine an actual coil parameter of the mounted second RF coil, and determine whether a difference between the actual coil parameter and the coil parameter is less than a difference threshold. If the difference is less than the difference threshold, the processing device 140 may determine that the mounted second RF coil satisfies the coil parameter. If the difference is larger than or equal to the difference threshold, the processing device 140 may determine that the mounted second RF coil does not satisfy the coil parameter, and the mounted second RF coil needs to be altered. The difference threshold may be determined based on the system default setting or set manually by the user.

In 1004, the processing device 140 (e.g., the obtaining module 920) may obtain PET data and MR data collected by the medical device in a scan of the subject under the one or more scanning parameters. The PET data may be collected by the PET detector module, and the MR data may be collected by the RF coil.

In some embodiments, after the one or more scanning parameters are determined, the PET imaging agent may be injected into the subject manually or automatically (e.g., through a mechanical arm), and the subject may be asked to hold the scanning posture. Then, the processing device 140 may direct the medical device to scan the subject under the one or more scanning parameters. In some embodiments, the processing device 140 may further determine whether the PET imaging agent is injected into the subject. For example, the processing device 140 may determine that the PET imaging agent is injected into the subject based on an instruction indicating that the PET imaging agent has be injected into the subject.

In some embodiments, the PET detector module may detect radiation photons emitted from the subject during the scan, and collect the PET data. The RF coil may receive MR signals related to the subject during the scan, and collect the MR data. Since the RF coil include a first RF coil and the second RF coil, the RF coil may receive two sets of MR signals. That is, the MR data may include two sets of MR data.

In some embodiments, the processing device 140 may obtain the PET data and the MR data from the medical device or a storage device that store the PET data and the MR data.

In 1006, the processing device 140 (e.g., the generation module 930) may generate, based on the PET data and the MR data, a fused image of the subject.

The fused image may refer to an image that fuses information of a plurality of images. As used herein, the fused image may be an image that fuses information of an MR image of the subject and information of a PET image of the subject. The fused image may provide more accurate and comprehensive anatomical and functional information of the subject than a signal-modality image (e.g., the PET image, the MR image, etc.), thereby improving the accuracy of the diagnosis.

In some embodiments, the processing device 140 may generate the PET image based on the PET data and generate the MR image based on the MR data. Since the MR data includes two sets of MR data, the processing device 140 may generate two independent and parallel MR images, and fuse the two independent and parallel MR images to the MR image. Alternatively, the processing device 140 may fuse the two sets of MR data, and generate the MR image based on the fused MR data. Accordingly, the processing device 140 may generate the fused image by fusing the PET image and the MR image. For example, the processing device 140 may extract information (e.g., anatomical information) of the MR image, and fuse the information of the MR image to the PET image to generate the fused image. As another example, the processing device 140 may extract information (e.g., functional information) of the PET image, and fuse the information of the PET image to the MR image to generate the fused image. As yet another example, the processing device 140 may generate the fused image by inputting the MR image and the PET image into an image fusion model (e.g., a trained machine learning model).

In some embodiments, the processing device 140 may preprocess the PET image and the MRI before the generation of the fused image. Exemplary preprocessing operations may include image transformation, uniformization, image enhancement, image denoising, image segmentation, image registration, or the like, or any combination thereof.

In some embodiments, the processing device 140 may post-process the fused image. Exemplary post-processing operations may include image enhancement, image denoising, image segmentation, smoothing, or the like, or any combination thereof.

In 1108, the processing device 140 (e.g., the determination module 910) may determine, based on the fused image, a position of a lesion of the subject.

In some embodiments, the processing device 140 may determine the position of the lesion of the subject automatically. For example, the processing device 140 may perform lesion positioning through an image segmentation technique. Exemplary image segmentation techniques may include an active contour algorithm, a semi-active contour algorithm, a graph-cut-based contour algorithm, a super-pixel-based segmentation algorithm, a Bayes-theory-based segmentation algorithm, an algorithm based on a machine learning model, or the like, or any combination thereof. In some embodiments, the user (e.g., a doctor, a technician) may determine the position of the lesion of the subject. For example, the user may delineate an outline of the lesion through an input device.

In some embodiments, the processing device 140 may determine the position of the lesion of the subject based on the fused image and a reference image. For example, a computed tomography (CT) image may be obtained before the simulation, and the processing device 140 may register the CT image and the fused image to determine the position of the lesion of the subject and generate a treatment plan.

In some embodiments, the processing device 140 may adjust the treatment plan based on the fused image. For example, the processing device 140 may obtain an evaluation of the simulation based on the fused image, and adjust the treatment plan based on the evaluation.

Merely by way of example, a PET-MR device may be used to determine a position of a lesion of a subject.

In some embodiments, the processing device 140 may determine one or more scanning parameters of the PET-MR device based on the subject. For example, the processing device 140 may obtain scanning information (e.g., a scanning protocol) of the subject, and determine the one or more scanning parameters (e.g., a type and/or a dose of a PET imaging agent) of the medical device based on the scanning information. In some embodiments, the processing device 140 may determine a scanning posture of the subject on the medical device based on the scanning information of the subject.

In some embodiments, the processing device 140 may further determine whether the PET imaging agent is injected into the subject. For example, after the one or more scanning parameters are determined, and the second RF coil is mounted in the medical device, the PET imaging agent may be injected into the subject manually or automatically, and the processing device 140 may determine whether the PET imaging agent is injected into the subject. In response to determining that the PET imaging agent is injected into the subject, the subject may be asked to hold the scanning posture. Then, the processing device 140 may direct the medical device to scan the subject under the one or more scanning parameters.

In some embodiments, after the subjected is scanned, the processing device 140 may obtain PET data and MR data from the PET-MR device or a storage device that store the PET data and the MR data. Further, the processing device 140 may generate a fused image of the subject based on the PET data and the MR data, and determine the position of the lesion of the subject based on the fused image.

According to some embodiments of the present disclosure, the second RF coil may be integrated in the medical device or detachably mounted in the medical device (disposed on the inner surface of the housing). Therefore, the second RF coil may be fixed to the medical device before the subject enters the scanning tunnel of the medical device or before the subject is injected the PET imaging agent, which can simplify the scanning process, save the scanning time, and increase the efficiency of the medical device. At the same time, the user may not need to mount the second RF coil after the subject is injected the PET imaging agent, so that the user may not be exposed to the PET imaging agent, which can reduce or eliminate the radiation damage to the user. Further, the second RF coil is integrated in the medical device or disposed on the inner surface of the housing without using a support to support the second RF coil around the subject. In this way, the RF coil and the second RF coil may be avoided from touching the surface of the subject to prevent muscle shape changes and organ displacement of the subject, a space of the scanning tunnel can be saved to allow the subject to hold various postures required for the simulation, thereby improving the accuracy of the simulation.

In addition, by introducing the second RF coil, the medical device may acquire more MR signals related to the subject than a medical device that only includes the first RF coil, which can improve a signal-to-noise ratio and imaging quality of MR imaging. Further, since the signal-to-noise ratio of MR imaging is improved, a thickness of the first RF coil and/or the second RF coil can be reduced, so that attenuations of the radiation photons emitted from the subject may be reduced, thereby improving the imaging quality of PET imaging. Therefore, the imaging quality of the medical device may be improved and the accuracy and the image quality of the fused image may be improved, which, in turn, improves the precise of subsequent diagenesis and treatment planning performed based on the fused image.

It should be noted that the descriptions of the process 1000 are provided for the purposes of illustration, and are not intended to limit the scope of the present disclosure. For persons having ordinary skills in the art, various variations and modifications may be conducted under the teaching of the present disclosure. However, those variations and modifications may not depart from the protection of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by the present disclosure, and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, device, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A medical device, comprising:
a housing that forms a scanning tunnel for accommodating a subject;
a positron emission tomography (PET) detector module arranged along a circumference of the scanning tunnel, the PET detector module being configured to detect radiation photons emitted from the subject during a scan; and
a radio frequency (RF) coil arranged along the circumference of the scanning tunnel, the RF coil being configured to receive magnetic resonance (MR) signals related to the subject during the scan, the RF coil including a first RF coil and a second RF coil, wherein
the first RF coil and the second RF coil are disposed coaxially around an axial direction of the scanning tunnel; and
a projection of the second RF coil along a radial direction of the scanning tunnel covers at least a portion of a gap of the first RF coil.

2. The device of claim 1, wherein the second RF coil is detachably mounted in the medical device.

3. The device of claim 2, wherein the first RF coil is disposed inside the housing, and the second RF coil is disposed on an inner surface of the housing.

4. The device of claim 3, wherein the medical device further includes one or more positioning components configured to fix the second RF coil on the inner surface of the housing.

5. The device of claim 4, wherein at least one of the one or more positioning components includes an interface used for current conduction and signal transmission of the second RF coil.

6. The device of claim 2, wherein
the housing includes a first housing and a second housing disposed coaxially around the axial direction of the scanning tunnel,
the second housing forms the scanning tunnel,
the first RF coil is disposed inside the first housing, and
the second RF coil is disposed inside the second housing.

7. The device of claim 2, wherein a coil parameter of the second RF coil is determined based on a scanning condition of the subject, the coil parameter of the second RF coil including at least one of a location, a size, a thickness, or a type of the second RF coil.

8. The device of claim 1, wherein the first RF coil and the second RF coil are integrated in the medical device.

9. The device of claim 1, wherein the second RF coil includes a preamplifier disposed outside a field of view (FOV) of the PET detector module.

10. A method, implemented on a computing device having at least one processor and at least one storage device, the method comprising:
determining, based on a subject to be scanned, one or more scanning parameters of a medical device, the medical device including a housing, a positron emission tomography (PET) detector module, and a radio frequency (RF) coil, wherein
the housing forms a scanning tunnel for accommodating the subject,
the PET detector module is arranged along a circumference of the scanning tunnel; and
the RF coil is arranged along the circumference of the scanning tunnel, the RF coil including a first RF coil and a second RF coil, the first RF coil and the second RF coil being disposed coaxially around an axial direction of the scanning tunnel, and a projection of the second RF coil along a radial direction of the scanning tunnel covering at least a portion of a gap of the first RF coil;
obtaining PET data and magnetic resonance (MR) data collected by the medical device in a scan of the subject under the one or more scanning parameters, the PET data being collected by the PET detector module, the MR data being collected by the RF coil; and
generating, based on the PET data and the MR data, a fused image of the subject.

11. The method of claim 10, wherein the determining, based on a subject to be scanned, one or more scanning parameters of a medical device includes:
determining a scanning posture of the subject on the medical device.

12. The method of claim 10, wherein the second RF coil is fixed to the medical device before the subject is injected a PET imaging agent.

13. The method of claim 10, further comprising:
determining, based on the fused image, a position of a lesion of the subject.

14. A system, comprising:
at least one storage device including a set of instructions; and
at least one processor configured to communicate with the at least one storage device, wherein when executing the set of instructions, the at least one processor is configured to direct the system to perform operations including:
determining, based on a subject to be scanned, one or more scanning parameters of a medical device, the medical device including a housing, a positron emission tomography (PET) detector module, and a radio frequency (RF) coil, wherein
the housing forms a scanning tunnel for accommodating the subject,
the PET detector module is arranged along a circumference of the scanning tunnel; and
the RF coil is arranged along the circumference of the scanning tunnel, the RF coil including a first RF coil and a second RF coil, the first RF coil and the second RF coil being disposed coaxially around an axial direction of the scanning tunnel, and a projection of the second RF coil along a radial direction of the scanning tunnel covering at least a portion of a gap of the first RF coil;
obtaining PET data and magnetic resonance (MR) data collected by the medical device in a scan of the subject under the one or more scanning parameters, the PET data being collected by the PET detector module, the MR data being collected by the RF coil; and
generating, based on the PET data and the MR data, a fused image of the subject.

15. The system of claim 14, further comprising:
determining, based on the fused image, a position of a lesion of the subject.

16. The system of claim 14, wherein the second RF coil is detachably mounted in the medical device.

17. The system of claim 16, wherein the first RF coil is disposed inside the housing, and the second RF coil is disposed on an inner surface of the housing.

18. The system of claim 17, wherein the medical device further includes one or more positioning components configured to fix the second RF coil on the inner surface of the housing.

19. The system of claim 16, wherein a coil parameter of the second RF coil is determined based on a scanning condition of the subject, the coil parameter of the second RF coil including at least one of a location, a size, a thickness, or a type of the second RF coil.

20. The system of claim 14, wherein the first RF coil and the second RF coil are integrated in the medical device.
